(19) United States
(12) Patent Application Publication
(10) Pub. No.: US 2021/0386886 A1
(43) Pub. Date: Dec. 16, 2021

(54) STERILIZATION DEVICE, SYSTEM, AND METHOD

(71) Applicant: Brian Awara Shukri, Kailua Kona, HI (US)

(72) Inventor: Brian Awara Shukri, Kailua Kona, HI (US)

(21) Appl. No.: 17/304,037

(22) Filed: Jun. 14, 2021

Related U.S. Application Data
(60) Provisional application No. 62/705,165, filed on Jun. 14, 2020.

(51) Int. Cl. A61L 2/10; A61L 2/26

(52) U.S. Cl. CPC A61L 2/10; A61L 2202/25; A61L 2202/24; A61L 2/26

(57) ABSTRACT

A sterilization apparatus and system are disclosed. The sterilization apparatus includes a housing, an ultraviolet an ultraviolet spectrum laser contained within the housing and designed to emit a sterilizing beam, a power source designed to power the laser, and a focusing element contained within the housing and designed to disperse the sterilizing beam to form at least one laser light plane for sterilizing an area. The sterilization system further includes a structure in or on which the housing is mounted.

### CROSS-REFERENCE TO RELATED APPLICATION

This application claims the benefit of priority of U.S. provisional application number 62/705,165, filed Jun. 14, 2020, the contents of which are herein incorporated by reference.

### BACKGROUND OF THE INVENTION AND DESCRIPTION OF THE RELATED ART

The present invention relates to sterilization methods and, more particularly, to a sterilization apparatus and method using a focused laser beam for targeted disbursement of electromagnetic energy below the wavelength of visible light.

Conventionally, electromagnetic energy with shorter wavelength than visible light (<400 nm), e.g., ultraviolet (UV) and x-ray, can be used to inactivate and destroy pathogens and thereby sterilize surfaces, air, other gases, liquid, and even penetrate solid matter. Energy of this wavelength has a sterilizing effect largely due to emission of ionizing radiation. Ionizing radiation, however, is hazardous to humans and other animals, as it is often carcinogenic and can cause burns and other radiation induced injury.

Conventional disinfecting devices require either proprietary air circulation systems, solid shielding, or cannot be used in a room without protective equipment. Other devices cannot inexpensively and dynamically decontaminate an area or object using UV light or other ionizing radiation in a non-toxic manner without adversely affecting people or other nonpathogens without their isolation. They cannot decontaminate all pathogens (e.g., low energy x-ray can sanitize bacteria and viruses while UV can only sanitize primarily viruses). Conventional sealed rooms do not allow rapid access and have longer periods of compromised containment by entry. Clothing and masks for preventing contamination are often bulky, impair visibility and can allow for cross-contamination. Many conventional solutions do not decontaminate a surface before a user comes in contact. Some related art is described below.

U.S. Pat. No. 5,768,853 and WO96/09775 describe the use of a UV radiation producing apparatus which deactivates microorganisms in food.

U.S. Pat. No. 3,817,703 discloses the use of a high energy density pulsed laser of unspecified wavelength to sterilize wine.

U.S. Pat. No. 5,232,367 discloses the use of a high power pulsed Nd:YAG laser in dental applications to sterilize the bacteria in a tooth cavity and accessory canals.

U.S. Pat. No. 3,941,670 teaches the use of an infrared CW laser to alter the biological activity of molecular species, including sterilization.

Chinese Pat. No. CN104368020A discloses the use of an ultra-Violet Laser disinfection system via collimation of laser and single-point rapid scanning mode, adds the laser intensity that can accurately control, and solves the uneven shortcoming with dosage instability of ultraviolet tube luminosity, effectively reaches effect of complete sterilizing.

As can be seen, there is a need for a sterilization method that alleviates all of the above-mentioned problems.

### SUMMARY OF THE INVENTION

In one aspect of the present invention, a sterilization apparatus comprises: a housing; an ultraviolet gas laser or laser diode contained within the housing and configured to emit a sterilizing beam; a focusing element (comprised of a laser optical assembly and motor) contained within the housing and configured to disperse the laser to form at least one plane of laser light to sterilize an area; and a power source configured to power the sterilizing beam and motor.

In another aspect of the present invention, a sterilization apparatus comprises: a housing; an ultraviolet spectrum gas laser or laser diode contained within the housing and configured to emit a sterilizing beam; a power source configured to power the ultraviolet gas laser or laser diode; a laser optical assembly contained within the housing and configured to disperse the sterilizing beam; and a motor to which the housing is mounted to rotate the device and function as a rotary laser thereby creating an effective plane of laser light.

These and other features, aspects and advantages of the present invention will become better understood with reference to the following drawings, description and claims.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a schematic view of an emitter of a first embodiment of the present invention

FIG. 2 is a schematic view of the embodiment of the present invention, mounted in a space;

FIG. 3 is a schematic view of an alternate mounting configuration of the embodiment of the present invention;

FIG. 4 is a schematic view of an alternate configuration of the emitter of the embodiment of the present invention;

FIG. 5 is a schematic view of a second embodiment of the present invention;

FIG. 6 is a schematic view of a third embodiment of the present invention;

FIG. 7 is a schematic view of a fourth embodiment of the present invention; and

FIG. 8 is a schematic view of a fifth embodiment of he present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, a sterilization apparatus and system are disclosed. The sterilization apparatus includes a housing, an ultraviolet spectrum laser diode or gas laser contained within the housing and designed to emit a sterilizing beam, a power source designed to power the laser, and a focusing element contained within the housing and designed to disperse the sterilizing beam to form at least one laser light plane for sterilizing an area. The sterilization apparatus further includes a structure in or on which the housing is mounted.

Embodiments of the present invention employ a laser to precisely focus sterilizing energy away from an unintended target, such as a human, so that pathogens can be sterilized safely. Using a focused laser, higher amplitude energy could be utilized than what would otherwise be deemed safe in the environment. A proximity sensor, such as a preceding laser and photoelectric switch, may also be used to deactivate the energy source and prevent accidental or unintended exposure. Conventional UV light bulbs emit unfocused or imprecisely focused energy. As previously mentioned, UV light is an extremely effective sterilization method, but it is also a carcinogen and exposure is a concern. UV-C wavelength (200 to 280 nm) is particularly effective as a germicidal. The present invention can focus continuous or pulsed energy to a point, line, or plane shape in conjunction with a laser optical assembly and/or a motor. Consequently, embodiments of the present invention can decontaminate a room with people still in it, but with no risk of contact to the light source.

Embodiments of the present invention are very versatile, as highly precise focusing of ionizing energy has many benefits. Inherent versatility of the present invention, as herein described, is an improvement when compared to existing solutions. Embodiments of the present invention can be used to decontaminate an area with people still in it, but with little if any risk of contact to the energy source. Embodiments of the present invention, either alone, or with multiple units, can be used to discreetly isolate a portion of a room, structure, or living being from pathogens. For sterilization of airborne pathogens in an occupied space, embodiments of the present invention can be retrofitted to an existing air circulation method, decreasing cost of installation and maintenance. Instead of a handheld or piloted decontamination unit that requires a person to aim or deploy the device at an area to be sterilized, this device can be mounted as a fixture and its use can be automated.

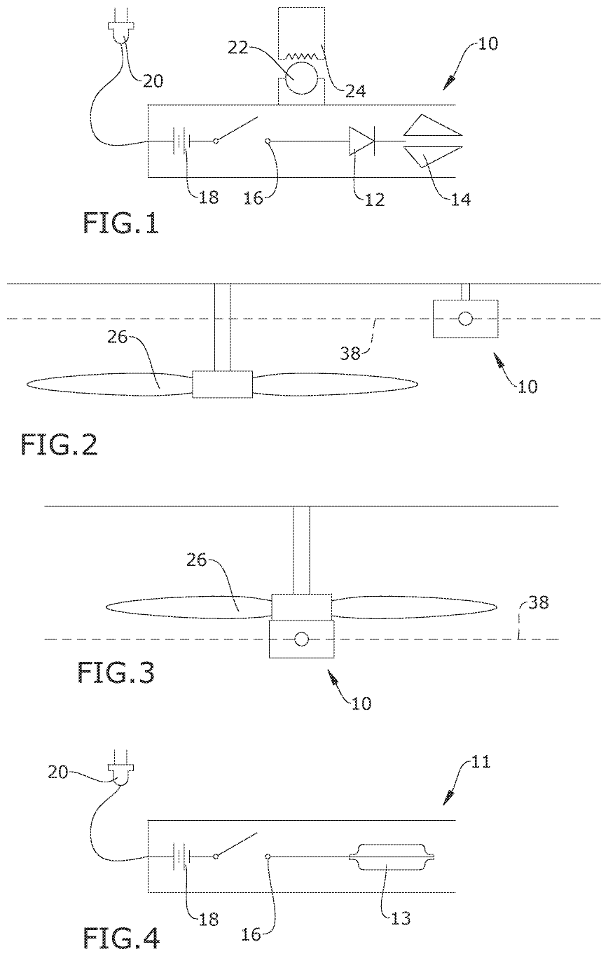

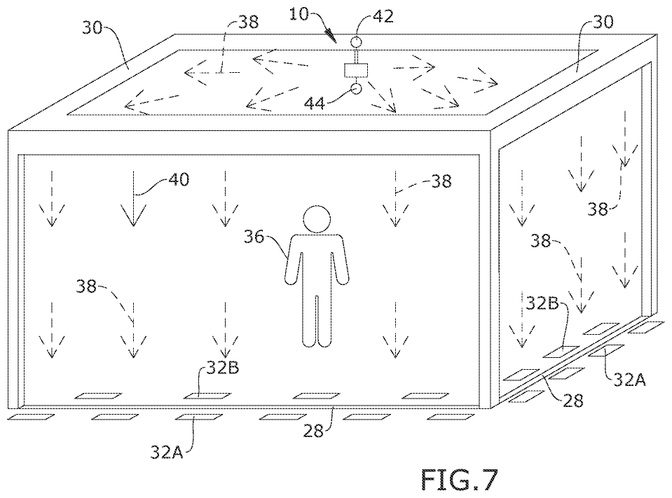

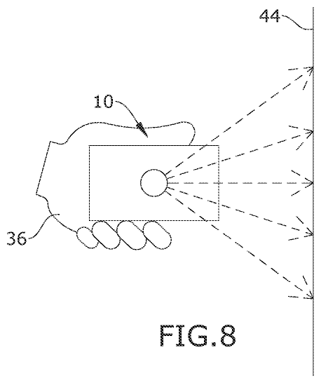

Referring now to FIGS. 1-8, certain embodiments of the present invention described herein include a UV spectrum laser emitter 10 with a laser diode 12, a focusing element 14 (while the term “focusing” is used, it is to be understood that dispersing the laser light is also considered to be a form of focusing or directing the light), such as a laser optical assembly (e.g., Powell lens, prism and/or focus mirrors 14) and/or motor, and a power source 18 (e.g., a battery or solar cell). As shown in FIG. 1, the laser emitter 10 may further include a switch 16 for selective operation of the emitter 10 (e.g., it can turn the emitter 10 on and off, as well as modulate the amplitude of the laser), and a plug 20 to provide wired power to the emitter 10. The switch 16 may, in certain embodiments, be activated by a sensor, such as a proximity sensor described in greater detail with respect to FIG. 7.

As shown in FIG. 1, the laser diode 12, focusing element 14, switch 16, and power source 18 may be provided in a housing (which may be custom configured to match the design of a space the emitter is installed in). In certain embodiments, such as that of FIGS. 1 and 2, the emitter 10 may further include a mount 24 that is rotated by an adjustment motor 22 (which may be provided on a three-axis gimbal which further enhances range of motion of the emitter 10) at fast speeds, e.g., 1100 revolutions per minute (RPM). Rotation of the motor 22 creates a UV/UV-C light plane (referred to in the art as a rotary laser). 38. The mount 24, in certain embodiments, may include a magnet for attachment to ferromagnetic surfaces. Further, a plurality of emitters 10 may be used in tandem to create multiple simultaneous sterilization planes, as needed.

Alternatively (or in addition to), and as shown in FIG. 3, the emitter 10 may be mounted on a fan 26 for rotation. Further, shielding material (not shown) may be optionally employed to protect a user 36 or other surface from exposure or scatter of energy from the emitter 10 to locations it is not intended. Making reference to FIG. 4, an alternative embodiment of the emitter 11 includes a gas laser 13 (e.g., Argon, fluoride). Those with skill in the art will appreciate that the gas laser 13 may be embodied in a similar manner as the embodiment shown in FIG. 1, with the laser diode 12 being substituted with the gas laser 13, and the gas laser sterilizing beam being manipulated by a corresponding focusing element 14.

In an exemplary use case (used for pathogen or hazardous/volatile material confinement), as shown in FIG. 7, the UV spectrum laser emitter 10 is supplied electricity by the power source 18 and manipulated (e.g., dispersed or focused) by the focusing element 14 to create a laser through the medium onto a target. The UV spectrum laser emitter's energy output is regulated and supplied by the power source 18 and switch 16 and dispersed or focused by the focusing element 14 to reach the intended target. The laser UV spectrum laser emitter 10 and focusing element 14, as previously described, can be mounted on a three-axis gimbal, which in turn can be moved by the adjustment motor 22. This arrangement increases range of motion of the emitter 10 and targeting versatility in any axis.

As mentioned above, FIG. 7 illustrates an exemplary scenario where the present invention may also be used for pathogen or hazardous/volatile material confinement. Certain embodiments of the present invention are capable of projecting a focused plane of energy. This plane may be further manipulated with mirrors/reflectors 30 to create a virtual room or enclosure using one or multiple units of the invention. For example, a plane 38 of UV laser beams may surround a hospital bed of an infected patient, as shown in FIG. 7. Advantageously, the beam of energy may be briefly interrupted if a caregiver were to enter and immediately be reenergized after entering or exiting the area. This may be accomplished utilizing proximity sensors 32A, 32B which, in certain embodiments, may be composed of preceding visible light emitting laser diodes 42, 44 positioned in parallel to the plane 38 of the UV laser beams and in concert with photoelectric switches.

More specifically, a sterilization system in accordance with the present invention may incorporate laser mirrors/reflectors 30, which, as shown by the dashed lines that illustrate the UV/UV-C light plane 38, redirects the light that forms the plane 38 to whatever form required to create a sterilized space where pathogens cannot enter. In such embodiments, UV light absorbing material 28 may be employed at terminal points of the path of the laser plane 38. Further, the sterilization system may include a plurality of proximity sensors 32A, 32B.

Even more specifically, one or more visible light laser diodes 42, 44 may be incorporated such that they are calibrated and aligned with the energy emission of the UV spectrum laser emitter 10. One or more visible light lasers 42, 44 create a visualization of the plane(s) of ionizing energy by emitting visible light 40. This provides a visual indication to individuals where the UV laser emitter is beaming its laser. In certain embodiments, the proximity sensors 32A, 32B may be embodied as photoelectric switches that, used in combination with the visible light lasers 42, 44, activate the switch 16 to shut off the emitter 10 if the visible light lasers 42, 44 are disrupted.

In different variations of the present invention as described above, the power source 18, the switch 16, the proximity sensors 32A, 32B, the shielding, the 3-axis gimbal, the visible light spectrum laser 42, 44 and the motor 22 for the gimbal may be placed within the emitter housing or be disposed outside the emitter housing depending on the application.

In accordance with aspects of the present invention, certain elements may be or may not be essential, depending on the application. For example, a motor 22 may be used to facilitate a rotary laser spinning at, for instance, 1100 revolutions per minute (RPM) creating an effective plane 38 of sanitizing energy while other axes move the laser around the space to be sanitized. If rotary motion is not required to create a plane 38 of sanitizing energy, the motor 22 may be eliminated.

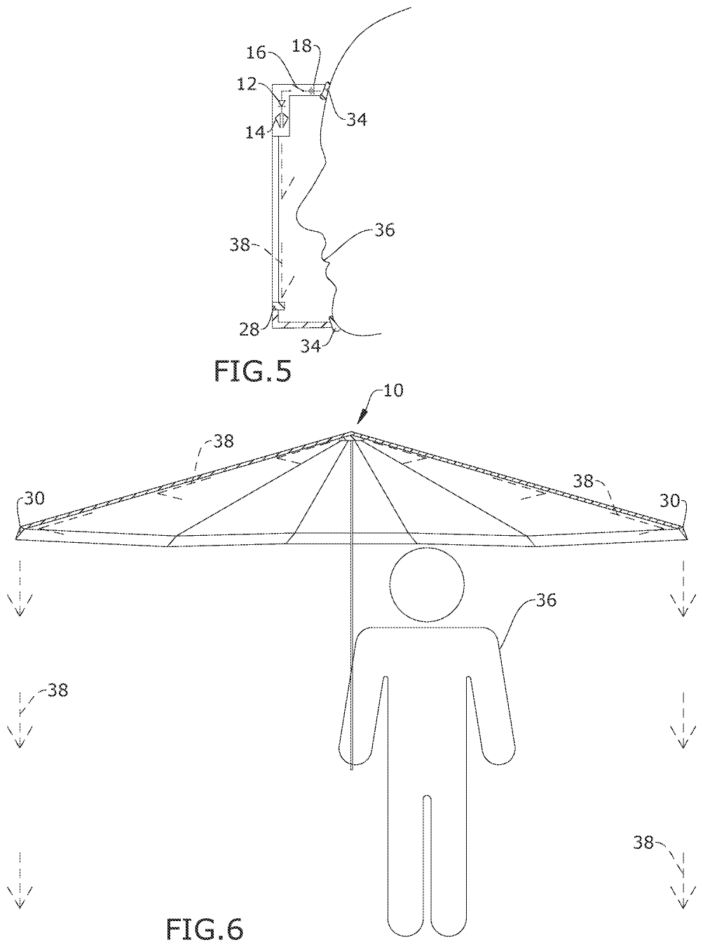

Embodiments of the present invention may also include a transparent respirator or personal pathogen enclosure, as shown in FIG. 5 and FIG. 6, respectively. As shown in FIG. 5, a laser beam may be projected as a plane 38 across or around a face and/or body of a user 36 and captured by shielding or non-reflective material 28. In such an embodiment, the previously described structure may be disposed in the respirator main body, and the main body be provided with cushioning material 34. Again, contact with the plane 38 of light may be prevented using a proximity sensor and switch. The personal pathogen enclosure of FIG. 6 may, for example, resemble an umbrella and form a substantially circular plane 38 of light surrounding a user 36. The emitted laser wavelength may also be optimized to ensure maximum safety dependent on the application.

As is readily apparent from the above discussion, the present invention may be used in a number of different ways. If sufficient power can be supplied to the present invention, it may be able to generate a magnetic field, especially if a laser beam is focused in a ring configuration. This magnetic field could then be used to contain or exclude certain types of hazardous or volatile material, such as radioactive plasma. A focused ring-shaped magnetic field may even potentially produce enough energy for magnetic resonance imaging with theoretically excellent field homogeneity and less mass than currently employed superconducting magnets. The present invention may be used in research to measure the effect of ionizing energy. The light can also be used to mark surfaces sensitive to energy with wavelengths shorter than visible light.

While methods of using the present invention would be readily apparent to those with skill in the art from the foregoing, a further method of using the present invention may include the following. It may be used for the sanitization of a space. For example, to sanitize the air in a room, as shown in FIG. 2, the emitter 10 can be suspended in the center of a room (e.g., 3 feet below the ceiling) using the mount 24. Power is supplied to the laser, projecting a circular plane 38 of UV laser light through the air onto the walls of the room using the focusing element 14. Air is then circulated though this plane 38 of UV light which continuously decontaminates pathogens that pass through it. The air may be circulated by using an already installed or inexpensively available method of air circulation, such as a ceiling fan. The sanitization device can also be mounted on a three-axis gimbal and the emitted plane of energy can be rotated along multiple axes with the motor assembly 22 for the gimbal and/or be mounted on a motorized track to target a larger sanitization area. As previously described, the present invention can be converted to a handheld sanitation device with the addition of a handle, shield and battery. In such an instance, a motorized gimbal can be utilized for stabilization to limit unintended exposure of emitted energy utilizing stabilization software for its control.

A method of making the present invention may include the following. A rudimentary prototype of the invention can be assembled by mounting a commercially available UV laser perpendicular to the axis of a motor. The motor is supplied sufficient power to spin at approximately 1100 RPM (known in the art as a rotary laser). Electrical power is regulated by a commonly available suitable power supply for both the UV emitting laser diode and motor. The contents may be mounted in a fabricated housing and mount. Specific refinements may then be made to include a commercially available motorized gimbal, laser optical assembly and shielding to aid in positioning and/or confinement of the sanitizing energy beam based on application. A photoelectric switch and visible laser(s) (both as a proximity sensor and as a visualization method for the sanitizing beam) may be used, as previously described. Software and a printed circuit board to control the function of the emitter(s) 10 and system may also be incorporated.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein.

While apparatuses and methods are described in terms of “comprising,” “containing,” or “including” various components or steps, the apparatuses and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A sterilization apparatus comprising:
a housing;
an ultraviolet spectrum gas laser or laser diode contained within the housing and configured to emit a sterilizing beam;
a power source configured to power the ultraviolet spectrum gas laser or laser diode; and
a focusing element contained within the housing and configured to disperse the sterilizing beam to form at least one laser light plane configured to sterilize an area.

2. The sterilization apparatus of claim 1, further comprising a mount for mounting the housing to a surface.

3. The sterilization apparatus of claim 2, wherein the mount comprises a motor for rotating the housing.

4. The sterilization apparatus of claim 1, further comprising a switch for turning the ultraviolet spectrum gas laser or laser diode on, off, and modulating an amplitude of the sterilizing beam.

5. A sterilization apparatus comprising:
a housing;
an ultraviolet spectrum gas laser or laser diode contained within the housing and configured to emit a sterilizing beam; and
a focusing element comprising a laser optical assembly and a motor, the focusing element being configured to disperse the sterilizing beam to form at least one laser light plane configured to sterilize an area.

6. A sterilization system comprising:
the sterilization apparatus of claim 5; and
a structure in or on which the housing is mounted.

7. The sterilization system of claim 6, further comprising one or more reflectors configured to redirect the at least one laser light plane in a direction different than an initial path of travel.

8. The sterilization system of claim 6, further comprising an absorbing material configured to absorb the sterilizing beam.

9. The sterilization system of claim 6, further comprising photoelectric switches for selectively signaling a switch to turn off the ultraviolet spectrum laser.

10. The sterilization system of claim 6, wherein the structure is a respirator or a room.

11. The sterilization system of claim 6, further comprising a visible light emitting laser diode that is configured to emit a visible light laser that is configured to run substantially parallel to the sterilizing beam.

12. The sterilization system of claim 11, further comprising a power source configured to power the ultraviolet spectrum gas laser or laser diode, the visible light emitting laser diode, and the motor.
